SEQUENCE LISTING (14 sequences)

SEQ ID NO: 1 - DNA, 20 bp, Artificial Sequence
  synthetic
gcccatcctctgtgactcat

SEQ ID NO: 2 - DNA, 20 bp, Artificial Sequence
  synthetic
aggccacaggtattttgtcg

SEQ ID NO: 3 - DNA, 22 bp, Artificial Sequence
  synthetic
cactcacctgctgctactcatt

SEQ ID NO: 4 - DNA, 20 bp, Artificial Sequence
  synthetic
tctggacccattccttcttg

SEQ ID NO: 5 - DNA, 20 bp, Artificial Sequence
  synthetic
tctactgaacttcggggtga

SEQ ID NO: 6 - DNA, 20 bp, Artificial Sequence
  synthetic
cacttggtggtttgctacga

SEQ ID NO: 7 - DNA, 20 bp, Artificial Sequence
  synthetic
tgcactaccaaagccacaag

SEQ ID NO: 8 - DNA, 20 bp, Artificial Sequence
  synthetic
tgggaagtgggtgcagttat

SEQ ID NO: 9 - DNA, 24 bp, Artificial Sequence
  synthetic
atgctcacactgacatcaacactc

SEQ ID NO: 10 - DNA, 23 bp, Artificial Sequence
  synthetic
ctcttccatcaccttgccaatcc

SEQ ID NO: 11 - DNA, 22 bp, Artificial Sequence
  synthetic
aagccagagtccttcagagaga

SEQ ID NO: 12 - DNA, 20 bp, Artificial Sequence
  synthetic
ggaaattggggtaggaagga

SEQ ID NO: 13 - DNA, 21 bp, Artificial Sequence
  synthetic
tcgttgggcgtctgaaggcaa

SEQ ID NO: 14 - DNA, 20 bp, Artificial Sequence
  synthetic
cggaagcactggcggcacat

(19) United States
(12) Patent Application Publication
(10) Pub. No.: US 2022/0133831 A1
(43) Pub. Date: May 5, 2022

(54) Sarcopoterium Spinosum Extract for Treating Inflammation

(71) Applicant: Ariel Scientific Innovations Ltd., Ariel (IL)

(72) Inventors: Tovit ROSENZWEIG, Kedumim (IL); Konstantin ROZENBERG, Rosh Haain (IL)

(21) Appl. No.: 17/434,266

(22) PCT Filed: Feb. 26, 2020
(86) PCT No.: PCT/IL2020/050215

§ 371 Date: Aug. 26, 2021

Related U.S. Application Data
(60) Provisional application No. 62/811,100, filed on Feb. 27, 2019.

(51) Int. Cl. A61K 36/73; A23L 33/105; A23L 33/00; A61P 29/00; A61P 1/00

(52) U.S. Cl. CPC A61K 36/73; A23L 33/105; A23V 2002/00; A61P 29/00; A61P 1/00; A23L 33/40

(57) ABSTRACT

The present invention provides a Sarcopoterium spinosum extract (SSE) for use in preventing or treating inflammation in a subject, and methods for treating inflammation in a subject by administering Sarcopoterium spinosum extract (SSE).

### FIELD OF INVENTION

The present invention relates to use of Sarcopoterium spinosum extract for treating inflammation.

### BACKGROUND OF THE INVENTION

Inflammation is part of the complex biological response of body tissues to harmful stimuli, such as pathogens, damaged cells, or irritants, and is a protective response involving immune cells, blood vessels, and chemical mediators. The function of inflammation is to eliminate the initial cause of cell injury, clear out necrotic cells and tissues damaged from the original insult and the inflammatory process, and initiate tissue repair. Inflammation is a generic response, and therefore it is considered as a mechanism of innate immunity.

Inflammation can be classified as either acute or chronic. Acute inflammation is the initial response of the body to harmful stimuli and is achieved by the increased movement of plasma and leukocytes (especially granulocytes) from the blood into the injured tissues. A series of biochemical events propagates and matures the inflammatory response, involving the local vascular system, the immune system, and various cells within the injured tissue. Prolonged inflammation, known as chronic inflammation, leads to a progressive shift in the type of cells present at the site of inflammation, such as mononuclear cells, and is characterized by simultaneous destruction and healing of the tissue from the inflammatory process. While inflammation is a vital process, and has a major role in the innate immunity, imbalance in the regulation of the inflammatory process might lead to sub-acute, chronic inflammation, which accompanies and is involved in the pathology of various chronic diseases.

Many pathologies involving inflammation exist, and many different types of inflammation exist. Examples for disorders involving inflammation are asthma, autoimmune diseases, inflammatory bowel diseases, dermatitis, and even cancer. Additionally, inflammation is involved in metabolic syndrome disorders, including insulin resistance, atherosclerosis and nonalcoholic steatohepatitis (NASH).

Chronic inflammation plays a special role in metabolic syndrome disorders, as it worsens insulin resistance by the action of pro-inflammatory cytokines, mainly TNFα, IL6 and IL1β. These cytokines activate c-jun kinase (JNK) and NFκB signaling that interfere in insulin signaling and amplify the developing insulin resistance Inflammation is also a hallmark of the atherogenic process, in which lymphocytes and monocytes infiltrate the intima media of arteries as a consequence of lipid deposition in this tissue. Differentiation of the infiltrated monocytes toward the proinflammatory phenotype leads to the secretion of cytokines and proteases that promotes the progression of atherosclerosis and increases the risk of plaque rupture and acute coronary events. Lastly, inflammation is considered as one of the ‘second hits’ involved in the deterioration of hepatic steatosis toward NASH. Activation of Kupffer cells, the resident hepatic macrophages, by inflammatory stimuli or metabolic alterations towards M1 macrophages is a critical event in the progression of NASH.

Accordingly, an intense effort is made in exploring novel anti-inflammatory therapies. Considering the potential adverse effects of unbalanced attenuation of the immune response, the development of anti-inflammatory therapies with a high safety profile is challenging. The plant kingdom includes a huge number of natural compounds with a wide range of bioactivity, which can be used for the treatment of various diseases, including inflammatory disorders. So far, several natural compounds have been verified to exhibit anti-inflammatory properties, including epigallocatechin gallate (EGCG), quercetin, and lupeol.

Sarcopoterium spinosum (S. spinosum), a chamaephyte of the Rosaceae family, is an abundant local medicinal herb in the Mediterranean area. Ethno-pharmacological surveys report the use of S. spinosum root extract for the treatment of diabetes by traditional Bedouin medicinal practitioners (Ali-Shtayeh et al., 2000; Bachrach et al., 2007). In previous studies by the inventors, the beneficial effects of this herb in reducing blood glucose, ameliorating insulin resistance and lowering the severity of fatty liver in mouse models were demonstrated (Rozenberg and Rosenzweig, 2018; Rozenberg et al. 2014; Smirin et al., 2010; Al-Qura'n 2009).

### SUMMARY OF THE INVENTION

In one aspect, the present invention provides a Sarcopoterium spinosum extract (SSE) for use in preventing or treating inflammation in a subject.

In another aspect, the present invention provides a nutraceutical composition comprising a Sarcopoterium spinosum extract (SSE) for use in preventing or treating inflammation in a subject.

In still another aspect, the present invention provides a method for preventing or treating inflammation in a subject, comprising administering to said subject an extract of Sarcopoterium spinosum (SSE).

### BRIEF DESCRIPTION OF THE DRAWINGS

FIGS. 1A-1E show SSE attenuation of white adipose tissue (WAT) inflammation by S. spinosum extract (SSE) in C57BL/6J mice given standard diet (STD) or high-fat diet (HFD). Visceral adipose tissue was removed (n=3), and H&E staining was performed. Representative H&E staining is presented in FIG. 1A (left, middle and right: C57BL/6J mice fed with STD, HFD, and HFD+SSE, respectively). The presence of crown-like structures (CLS) was quantified in all fields of the sections (B), and adipocyte diameter was measured (C-D, n depicts the number of cells measured) In D: solid line—STD; dashed line—HFD; dotted line—HFD+SSE. Results are presented as the Mean±SEM, ****P<0.0001 by one-way Anova, followed by Bonferroni's post-test. Adipocytokine expression of various cytokines was measured as described in Methods (E) Solid black bars STD; gray bars—HFD; empty bars—HFD+SSE. *P<0.05, **P<0.005, ***P<0.0005 by two-way Anova, followed by Tukey's post-test.

FIGS. 2A-2D show SSE attenuation of white adipose tissue (WAT) inflammation by S. spinosum extract (SSE) in KK-Ay mouse model of insulin resistance. Visceral adipose tissue was removed (n=3), and H&E staining was performed. Representative H&E staining is presented in FIG. 2A (left, right: KK-Ay mice not fed, and fed, with SSE). The presence of crown-like structures (CLS) was quantified in all fields of the sections (B), and adipocyte diameter was measured (C-D, n depicts the number of cells measured) In D: solid line—−SSE; dashed line—+SSE. . Results are presented as the Mean±SEM, ****P<0.0001 by students t-test.

FIGS. 3A-3B show increased viability and affected cell morphology in RAW265.7 cells in response to SSE treatment. RAW264.7 cells were treated with lipopolysaccharide (LPS) or SSE for 24 h. A. Viability was measured by XTT assay. Results are presented as the Mean±SEM, *P<0.05, **P<0.005, ***P<0.001 by one-way Anova, followed by Bonferroni's post-test. B. Micrographs of cells were performed using bright field Olympus imaging system, ×20 magnification. Upper two: left—control; right—LPS; lower two: left—SSE; right—LPS+SSE.

FIGS. 4A-4B show that SSE attenuated LPS-induced NO production and regulated mRNA expression of pro and anti-inflammatory genes in RAW264.7 cells. RAW264.7 cells were treated with LPS and/or SSE for 24 h. A. supernatant was collected and nitrite was measured by Griess reagent. B. mRNA was isolated and the expression of the indicated genes was measured as described in the methods. ΔΔCt means—normalized by the control. Results were normalized to the expression of the housekeeping gene, RPS29. *P<0.05, **P<0.005, ***P<0.0001 by one-way Anova, followed by Bonferroni's post-test.

FIGS. 5A-5D show that SSE stimulated the activation of MAPK, but not NFκB or Akt pathways in RAW264.7 cells. RAW264.7 cells were treated with LPS and/or SSE for 24 h. A. Nuclear proteins were extracted and NFκB activation was measured as described in the methods. ***P<0.0001 by one-way Anova, followed by Bonferroni's post-test. B-D. Western-blot analysis was performed as described in the methods. These are representative results of 3 independent experiments.

FIGS. 6A-6D show an immunomodulatory effect of SSE on bone marrow-derived macrophages (BMDM). BMDM were isolated and cultured as described in the methods. Cells were treated with LPS and/or SSE for 24 hr. A. micrographs of cells were performed by bright field Olympus imaging system. Upper two: left—control; right—LPS; lower two: left—SSE; right—LPS+SSE. B. NO synthesis was measured by Griess reagent as described in the methods. C. mRNA was isolated and expression of selected genes was measured by RT-PCR as described in the methods. Results were normalized to the expression of the housekeeping gene RPS29, and are presented as the Mean±SEM. *P<0.05, **P<0.005, ***P<0.001 ****P<0.0001 compared to control, or as indicated in graphs, by one-way Anova, followed by Bonferroni's post-test. D. Western-blot analysis was performed as described in the methods. These are representative results of 3 independent experiments.

### DETAILED DESCRIPTION OF THE INVENTION

In the present invention, the inventors demonstrate the immunomodulatory effects of Sarcopoterium spinosum extract (SSE) in several different experimental models: in adipose tissue under pro-inflammatory metabolic stress (in two different mouse models), in RAW264.7 macrophages, and in bone marrow-derived macrophages (BMDM). The results of this study, obtained from all experimental setups, indicate that SSE exerts immunomodulatory effects and affects inflammatory response.

The inventors have shown here that SSE affects the phenotype of macrophages, polarizing them to favor an anti-inflammatory profile, which points to a direct immunomodulatory function of SSE. Macrophages are key players in the inflammatory response, having three major function; antigen presentation, phagocytosis, and immunomodulation through production of various cytokines and growth factors. Macrophages play a critical role in the initiation, maintenance, and resolution of inflammation. Macrophages are located in most body tissues, either as resident tissue-specific macrophages, or as a result of the recruitment of monocytes/macrophages to the tissue in response to local inflammatory stimuli. Both the recruited and the resident macrophages are highly sensitive to micro-environmental cues, both physiological and pathological, leading to the activation of various complexed and interacting endogenous signaling pathways that direct M1/M2 polarization of the cells, enabling these cells to adopt a distinct functional phenotype. Moreover, it was shown that M1/M2 polarization status can be rapidly induced or reversed, enabling the high plasticity of these cells. Accordingly, agents that might direct the programming of macrophages from MΦ to M2, or the reprogramming from M1 to M2, might be of high therapeutic potential in pathologies which involve chronic inflammation. The results presented herein suggest that SSE can direct macrophages toward a M2-like, anti-inflammatory, profile.

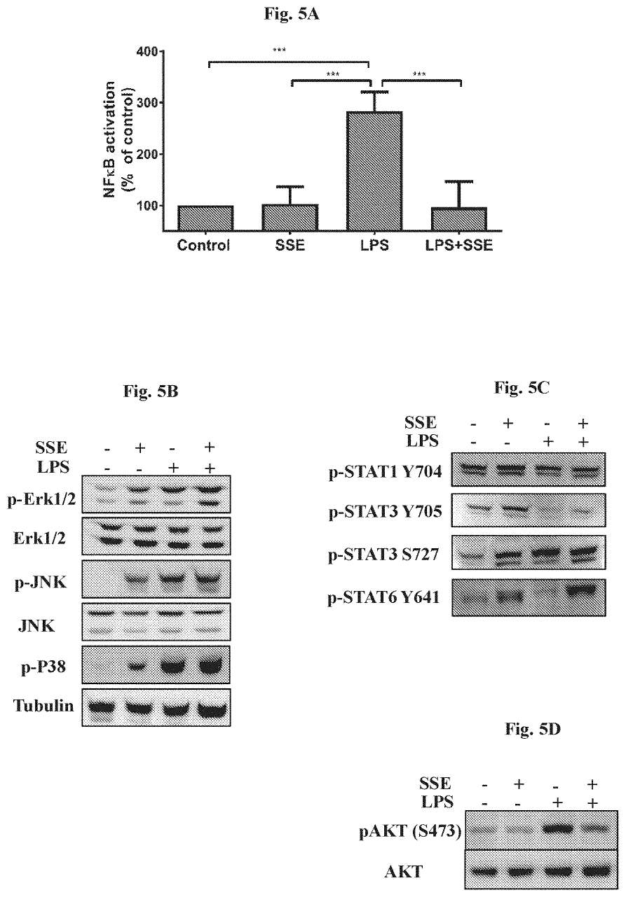

The results below show that in RAW264.7 cells, lipopolysaccharide (LPS), a ligand of the TLR4, activates NFκB and MAPK pathways, which are stimulatory signals for inflammation, in agreement with previously reported data. The PI3K-Akt pathway was also activated by LPS, as expected. In contrast, SSE abrogated the activation of these proinflammatory signals, mainly NFκB (FIG. 5A), which is a central player in M1 polarization, and induced the phosphorylation of STAT3 and STAT6 (FIG. 5C), which are known to be activated by cytokines such as IL4 and IL10, and to increase M2 macrophage polarization. Binding of such cytokines to their receptors activates the Janus kinase (JAK) family, which phosphorylates the receptor, enabling the binding of STAT3, its tyrosine phosphorylation, dimerization and subsequently its translocation to the nucleus and transcriptional activation. STAT3 tyrosine phosphorylation is an event associated with the activation of anti-inflammatory signals. Similarly, tyrosine phosphorylation of STAT6 is known to be facilitated by anti-inflammatory cytokines such as IL4/13. Thus, the data presented below, demonstrating the induction of tyrosine phosphorylation of STAT3/6 by SSE (and not by LPS), indicates the anti-inflammatory effect of the SSE in RAW264.7 macrophages.

Accordingly, in one aspect, the present invention provides a Sarcopoterium spinosum (S. spinosum) extract (SSE) for use in preventing or treating inflammation in a subject.

In some embodiments, the inflammation is acute. In some embodiments, the inflammation is chronic. In some embodiments, the inflammation is sub-acute inflammation.

An acute inflammation is the initial response of the body to harmful stimuli and is achieved by the increased movement of plasma and leukocytes from the blood into the injured tissues. Acute inflammation may result from tissue injury or damage. The tissue injury or damage may result from an infection, a hypersensitivity reaction, a physical or a chemical agent, or tissue necrosis.

Chronic (prolonged) inflammation leads to a progressive shift in the type of cells present at the site of inflammation, such as mononuclear cells, and is characterized by simultaneous destruction and healing of the tissue.

Sub-acute inflammation is the phase between acute and chronic inflammation, lasting about 2-6 weeks.

Many diseases and conditions are associated with inflammatory processes. Below are examples for diseases, disorders and conditions associated with inflammatory processes.

Inflammatory bowel disease (IBD) is a group of inflammatory conditions of the colon and small intestine. Crohn's disease and ulcerative colitis are the principal types of inflammatory bowel disease. Crohn's disease affects the small intestine and large intestine, as well as the mouth, esophagus, stomach and the anus, whereas ulcerative colitis primarily affects the colon and the rectum.

Autoimmune diseases are conditions arising from an abnormal immune response to a normal body part. There are at least 80 types of autoimmune diseases and nearly any body part can be involved. Some common examples include asthma, celiac disease, IBD, multiple sclerosis, psoriasis, rheumatoid arthritis, and systemic lupus erythematosus.

Additional examples of diseases involving inflammation include bursitis, colitis, cystitis, dermatitis, encephalitis, gingivitis, hepatitis, meningitis, myelitis, nephritis, neuritis, periodontitis, pharyngitis, phlebitis, prostatitis, rhinitis, sinusitis, tendonitis, testiculitis, tonsillitis, urethritis, vasculitis, vaginitis, and metabolic syndrome associated disorders such as insulin resistance, atherosclerosis or nonalcoholic steatohepatitis.

Accordingly, in some embodiments, the inflammation is associated with a disease, disorder or condition selected from bursitis; colitis; cystitis; dermatitis; encephalitis; gingivitis; hepatitis; meningitis; myelitis; nephritis; neuritis; periodontitis; pharyngitis; phlebitis; prostatitis; rhinitis; sinusitis; tendonitis; testiculitis; tonsillitis; urethritis; vasculitis; vaginitis; alcoholic steatohepatitis; an inflammatory bowel disease (IBD) such as ulcerative colitis or Chron's disease; an autoimmune disease such as asthma, celiac disease, multiple sclerosis, psoriasis, rheumatoid arthritis, or systemic lupus erythematosus; and a metabolic syndrome associated disorder such as insulin resistance, atherosclerosis or nonalcoholic steatohepatitis.

In some embodiments, the disease, disorder or condition is selected from dermatitis; hepatitis; alcoholic steatohepatitis; an inflammatory bowel disease such as ulcerative colitis or Chron's disease; an autoimmune disease such as rheumatoid arthritis or systemic lupus erythematosus; and a metabolic syndrome associated disorder such as insulin resistance, atherosclerosis or nonalcoholic steatohepatitis.

In some embodiments, the disease, disorder or condition is selected from dermatitis; alcoholic steatohepatitis; ulcerative colitis; Chron's disease; rheumatoid arthritis; and systemic lupus erythematosus.

In some embodiments, the disease, disorder or condition is selected from dermatitis; alcoholic steatohepatitis; ulcerative colitis; Chron's disease; rheumatoid arthritis; systemic lupus erythematosus; insulin resistance; and atherosclerosis.

In some embodiments, the disease, disorder or condition is selected from dermatitis; ulcerative colitis; Chron's disease; rheumatoid arthritis; and systemic lupus erythematosus.

In some embodiments, the subject is suffering from metabolic syndrome or a metabolic syndrome-related condition.

In some embodiments, the subject is not suffering from metabolic syndrome.

In some embodiments, the subject is not suffering from a metabolic syndrome-related condition.

Metabolic syndrome-related conditions, as defined herein, include obesity, high blood pressure, high blood sugar, high serum triglycerides, and low serum high-density lipoprotein (HDL). Metabolic syndrome is defined herein as a cluster of at least three of the above metabolic syndrome-related conditions.

In some embodiments, the subject is not suffering from atherosclerosis. In some embodiments, the subject is not suffering from fatty liver. In some embodiments, the subject is not suffering from nonalcoholic steatohepatitis (NASH).

In some embodiments, the subject is not diabetic or pre-diabetic.

Having diabetes or being diabetic is defined herein as having Hemoglobin A1C>6% or blood glucose level≥125 mg/dL. The term “diabetic” includes individuals having Type I diabetes or Type II diabetes. The term “pre-diabetic” relates to individuals having blood glucose level between 100 and 125 mg/dL.

Extracts of S. Spinosum may be prepared from the whole plant, as well as from various parts of the plant. Traditionally, the roots of the plant have been used for preparing the extract.

In certain embodiments, the S. spinosum extract is an extract from the roots of the plant. In certain other embodiments, the S. spinosum extract is an extract of the fruits and/or leaves of the plant. In some embodiments, the S. spinosum extract is a whole plant extract.

In some embodiments, the S. spinosum extract is formulated for administration in liquid form, preferably in water. In certain embodiments, the S. spinosum extract is formulated in dry form, for example, as powder, a tablet or a capsule.

In some embodiments, the extract is obtained by boiling of the desired plant part, e.g. root, in water or another suitable solvent, filtering, and optionally lyophilization to get a dry extract. In some embodiments, the extract is obtained by boiling S. Spinosum roots in water, filtering, and optionally lyophilization to obtain a dry extract.

In some embodiments, the S. spinosum extract is suitable for administration that is not topical. In some embodiments, the S. spinosum extract is suitable for enteric administration.

In certain embodiments, the S. spinosum extract is suitable for oral administration.

The extracts of the present invention may be prepared by preparing tea, infusion, decoction, percolation, or by similar methods of extraction of chemicals from a plant. The extraction may be done in water, or in another appropriate solvent.

In another aspect, the present invention relates to a pharmaceutical composition comprising an extract of Sarcopoterium spinosum as defined above, for use in preventing or treating inflammation as described above.

The pharmaceutical composition may be formulated with a pharmaceutically acceptable carrier or excipient. In certain other embodiments, the composition is formulated as a herbal composition, such as a herbal composition powder.

Pharmaceutical compositions for use in accordance with the present invention may be formulated in conventional manner using one or more physiologically acceptable carriers or excipients. The carrier(s) must be “acceptable” in the sense of being compatible with the other ingredients of the composition and not deleterious to the recipient thereof.

The following exemplification of carriers, modes of administration, dosage forms, etc., are listed as known possibilities from which the carriers, modes of administration, dosage forms, etc., may be selected for use with the present invention. Those of ordinary skill in the art will understand, however, that any given formulation and mode of administration selected should first be tested to determine that it achieves the desired results.

The pharmaceutical preparation for oral administration may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets, pills, lozenges, or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art.

Nutraceuticals are natural, bioactive chemical substances or extracts that provide numerous physiological benefits, including, inter alia, disease prevention and health promotion.

Accordingly, in yet another aspect, the present invention relates to a nutraceutical composition comprising an extract of Sarcopoterium spinosum as defined above, for use in preventing or treating inflammation as described above. In some embodiments, the inflammation is acute. In some embodiments, the inflammation is chronic.

In certain embodiments, the composition is a nutraceutical composition that may comprise other nutritional or dietary supplements such as vitamins, and/or one or more excipients that may be pharmaceutically acceptable and/or nutraceutical carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms. Each carrier must be acceptable in the sense of being compatible with the other ingredients of the formulation and not injurious to the patient. In certain embodiments, the composition is formulated as an oral formulation that may be liquid or solid, e.g., in the form of tablets, lozenges, capsules, syrup and the like.

In some embodiments, the pharmaceutical or the nutraceutical composition of the invention is used for preventing or treating inflammation associated with a disease, disorder or condition selected from bursitis; colitis; cystitis; dermatitis; encephalitis; gingivitis; hepatitis; meningitis; myelitis; nephritis; neuritis; periodontitis; pharyngitis; phlebitis; prostatitis; rhinitis; sinusitis; tendonitis; testiculitis; tonsillitis; urethritis; vasculitis; vaginitis; alcoholic steatohepatitis; an inflammatory bowel disease (IBD) such as ulcerative colitis or Chron's disease; an autoimmune disease such as asthma, celiac disease, multiple sclerosis, psoriasis, rheumatoid arthritis, or systemic lupus erythematosus; and a metabolic syndrome associated disorder such as insulin resistance, atherosclerosis or nonalcoholic steatohepatitis.

In some embodiments, the disease, disorder or condition is selected from dermatitis; hepatitis; alcoholic steatohepatitis; an inflammatory bowel disease such as ulcerative colitis or Chron's disease; an autoimmune disease such as rheumatoid arthritis or systemic lupus erythematosus; and a metabolic syndrome associated disorder such as insulin resistance, atherosclerosis or nonalcoholic steatohepatitis. In some embodiments, the disease, disorder or condition is selected from dermatitis; alcoholic steatohepatitis; ulcerative colitis; Chron's disease; rheumatoid arthritis; and systemic lupus erythematosus.

In some embodiments, the disease, disorder or condition is selected from dermatitis; alcoholic steatohepatitis; ulcerative colitis; Chron's disease; rheumatoid arthritis; systemic lupus erythematosus; insulin resistance; and atherosclerosis.

In some embodiments, the disease, disorder or condition is selected from dermatitis; ulcerative colitis; Chron's disease; rheumatoid arthritis; and systemic lupus erythematosus.

In some embodiments, the subject is suffering from metabolic syndrome or a metabolic syndrome-related condition.

In some embodiments, the subject is not suffering from metabolic syndrome.

In some embodiments, the subject is not suffering from a metabolic syndrome-related condition.

In some embodiments, the subject is not diabetic or pre-diabetic.

In some embodiments, the extract is an extract from the root of S. spinosum.

In some embodiments, the extract is in a liquid form. In some embodiments, the extract is in a dry form, such as powder, a tablet or a capsule.

According to another aspect, the present invention provides a method for preventing or treating inflammation in a subject, comprising administering to said subject an extract of Sarcopoterium spinosum (SSE) according to the invention as described above.

In some embodiments, the inflammation is acute. In some embodiments, the inflammation is chronic. In some embodiments, the inflammation is associated with a disease, disorder or condition selected from bursitis; colitis; cystitis; dermatitis; encephalitis; gingivitis; hepatitis; meningitis; myelitis; nephritis; neuritis; periodontitis; pharyngitis; phlebitis; pro statitis; rhinitis; sinusitis; tendonitis; testiculitis; tonsillitis; urethritis; vasculitis; vaginitis; alcoholic steatohepatitis; an inflammatory bowel disease (IBD) such as ulcerative colitis or Chron's disease; an autoimmune disease such as asthma, celiac disease, multiple sclerosis, psoriasis, rheumatoid arthritis, or systemic lupus erythematosus; and a metabolic syndrome associated disorder such as insulin resistance, atherosclerosis or nonalcoholic steatohepatitis.

In some embodiments, the disease, disorder or condition is selected from dermatitis; hepatitis; alcoholic steatohepatitis; an inflammatory bowel disease such as ulcerative colitis or Chron's disease; an autoimmune disease such as rheumatoid arthritis or systemic lupus erythematosus; and a metabolic syndrome associated disorder such as insulin resistance, atherosclerosis or nonalcoholic steatohepatitis.

In some embodiments, the disease, disorder or condition is selected from dermatitis; alcoholic steatohepatitis; ulcerative colitis; Chron's disease; rheumatoid arthritis; and systemic lupus erythematosus.

In some embodiments, the disease, disorder or condition is selected from dermatitis; alcoholic steatohepatitis; ulcerative colitis; Chron's disease; rheumatoid arthritis; systemic lupus erythematosus; insulin resistance; and atherosclerosis.

In some embodiments, the disease, disorder or condition is selected from dermatitis; ulcerative colitis; Chron's disease; rheumatoid arthritis; and systemic lupus erythematosus.

In some embodiments, the subject is suffering from metabolic syndrome or a metabolic syndrome-related condition.

In some embodiments, the subject is not suffering from metabolic syndrome.

In some embodiments, the subject is not suffering from a metabolic syndrome-related condition.

In some embodiments, the subject is not diabetic or pre-diabetic.

In some embodiments, the extract is an extract from the root of S. spinosum.

In some embodiments, the extract is in liquid form. In some embodiments, the extract is in a dry form, such as powder, a tablet or a capsule.

The determination of the doses of the active ingredient to be used for human use is based on commonly used practices in the art, and will be finally determined by physicians in clinical trials. An expected approximate equivalent dose for administration to a human can be calculated based on the in vivo experimental evidence disclosed herein below, using known formulas (e.g. Reagan-Show et al. (2007) Dose translation from animal to human studies revisited. The FASEB Journal 22:659-661). According to this paradigm, the adult human equivalent dose (mg/kg body weight) equals a dose given to a mouse (mg/kg body weight) multiplied with 0.081.

The S. spinosum extract may be administered once or more daily, such as twice daily, or three times daily, as needed. Alternatively, the S. spinosum extract may be administered less than once daily, such as once every two, three, or four days.

The term “carrier” refers to a diluent, adjuvant, excipient, or vehicle with which the active agent is administered. The carriers in the pharmaceutical composition may comprise a binder, such as microcrystalline cellulose, polyvinylpyrrolidone (polyvidone or povidone), gum tragacanth, gelatin, starch, lactose or lactose monohydrate; a disintegrating agent, such as alginic acid, maize starch and the like; a lubricant or surfactant, such as magnesium stearate, or sodium lauryl sulphate; and a glidant, such as colloidal silicon dioxide.

The term “treating” or “treatment” as used herein refers to means of obtaining a desired physiological effect. The effect may be therapeutic in terms of partially or completely curing a disease and/or symptom attributed to the disease. The term refers to inhibiting the disease, i.e. arresting its development; or ameliorating the disease, i.e. causing regression of the disease. This term also includes reversing or slowing the progression of disease activity or the medical consequences of the disease.

The term “preventing” as used herein relates to suspending, postponing, delaying or completely abolishing the appearance of a certain disorder, disease, or condition, or the appearance of symptoms associated with a certain disorder, disease, or condition. Further, the terms “preventing” and “prevention” refer to prophylactic use to reduce the likelihood of a disease, disorder, or condition to which such terms apply, or one or more symptoms of such disease, disorder, or condition. It is not necessary to achieve a 100% likelihood of prevention; it is sufficient to achieve at least a partial effect of reducing the risk of acquiring such disease, disorder, or condition.

It is noted that unless explicitly stated, any value recited in the claims is intended to encompass values of up to 10% above or below the recited value.

The invention will now be demonstrated by the following non-limiting examples.

### EXAMPLES

### Materials

Reagents and media for cell culture were obtained from Biological Industries (Beit Haemek, Israel), with lipopolysaccharide (LPS) purchased from Sigma. M-CSF was purchased from Peprotech (Israel). Primary antibodies were all obtained from Cell-Signaling Technology. Secondary antibodies were purchased from Jackson ImmunoResearch.

### Methods

S. Spinosum Extract Preparation

100 g fresh S. spinosum roots were cut into small pieces and boiled in 1 L of water for 30 min. The solutions were left for 3 h and the red supernatants were filtered to a sterile bottle without disturbing the pellet, and kept at 4° C. In the experiments indicated, extract was dried by lyophilization, giving a yield of 7 gr/L (0.7% w/v).

Adipose Tissue Inflammation

The protocol of the study was approved by the Committee on the Ethics of Animal Experiments of the University of Ariel. The Animal House in Ariel University operates in compliance with the rules and guidelines of the Israel Council for Research on Animals, based on the US NIH Guide for the Care and Use of Laboratory Animals. The mice were housed in an animal laboratory with a controlled environment of 20-24° C., 45-65% humidity, and a 12 h light/dark cycle. Animals had been anesthetized by ketamine+xylazine as required, and all efforts were made to minimize suffering. The study was performed on KK-Ay mice, a genetic model of type 2 diabetes (T2D), and on a model of diet-induced glucose intolerance, using high fat diet-fed C57bl/6J mice (HFD, 60% of total calories derived from fatty acids, 18.4% from proteins, and 21.3% from carbohydrates, Envigo, Teklad TD.06414). For these experiments, 6 week old male mice were separated into treatment groups, 8-10 mice each. KK-Ay male mice (Jackson Laboratories) were separated into 2 groups as follows: control-untreated mice and Sarcopoterium spinosum extract (SSE)-treated mice. The mice were given a standard (STD) diet (18% of total calories derived from fat, 24% from proteins, and 58% from carbohydrates. Envigo, Teklad TD.2018). C57bl/6J male mice (Envigo, Israel) were separated into 3 treatment groups, 8-10 mice each, as follows: control mice fed with STD or HFD, and HFD-fed mice supplemented with SSE. Average daily water consumption was measured, and the dry extract was reconstituted in this volume (5 ml in C57bl/6J, 15 ml in KK-Ay mice) in order to obtain the dose of 35 mg/day in C57bl/6J mice and 100 mg/day in KK-Ay mice). SSE solution was replaced every 2 days in order to avoid spoiling, starting at age 6 weeks till the age of 14-15 weeks.

Mice were anesthetized using ketamine+xylazine and euthanized by terminal bleeding followed by cervical dislocation. Adipose tissue was collected and preserved in 4% paraformaldehyde and at −80° C. for histological and molecular analyses

Histochemistry

White Adipose Tissue (WAT) was isolated and fixed in 4% paraformaldehyde and embedded in paraffin. Consecutive 4 μm sections were cut and stained with hematoxylin and eosin (H&E). Adipocyte diameter was measured using Image J software.

Adipocytokine Expression

Adipocytokine expression was measured in adipose tissue of c57bl/6J given STD, HFD or HFD+SSE obtained as described above. Protein lysate of the adipose tissue was prepared and adipocytokine expression was measured according to manufacturer's instructions using Mouse Adipokine Array Kit (R&D Systems, ARY013), enabling a simultaneous detection of 38 adipocytokines.

Cell Culture

In-vitro experiments were performed on RAW264.7 mouse macrophage cell line and on bone marrow-derived macrophages (BMDM). RAW264.7 cells were grown in high glucose DMEM containing 10% FBS, 2 mM 1-glutamine and 1% penicillin/streptomycin. For BMDM: C57bl/6J mice at age 10 weeks were purchased from Envigo, Israel. BMDMs were isolated from femur and tibia bones. Mice were sacrificed using a CO₂ chamber. Mice legs were cut off, while fur, flesh and muscle tissue were removed in sterile environment followed by disconnection of the tibia from the femur. Bone marrow content was washed out using a complete RPMI and 23G syringe followed by filtration of the supernatant through a 21G syringe. The cells were centrifuged at 450×g for 10 min at room temperature and the pellet was resuspended in RPMI containing 10% FBS, 1% penicillin/streptomycin, HEPES buffer (120 Mm) and M-CSF (50 μg/ml). The media was replaced every 3 days. The experiment was performed on the 7^{th} day of differentiation.

Cell Viability Assay

RAW264.7 cells were seeded (5×10⁴ cells/well) in 96-well plates, and incubated overnight followed by treatment with LPS (200 ng/ml) and/or SSE (70 μg/ml). Cell viability was determined by XTT assay (Biological Industries, Israel. Cat. No.: 20-300-1000) according to manufacturer instructions.

Real Time PCR

Total RNA was extracted from Raw264.7 cells using TRI reagent (Molecular Research Center, Inc. Cincinnati, Ohio) according to manufacturer instructions. RNA (4 μg) was reverse transcribed by oligo-dT priming (Stratascript 5.0 multi-temperature reverse transcriptase, Stratagene) according to manufacturer instructions. Real-time PCR amplification reactions were performed using SYBR Fast Universal Ready-mix Kit (Kappa Biosystems), using the MxPro QPCR instrument (Stratagene). Primers for real time PCR reactions were synthesized by Sigma, Israel. Primer sequences are listed in Table 1.

TABLE 1
primers list

| Gene | Forward | SEQ ID | Reverse | SEQ ID |
|---|---|---|---|---|
| IL-1β | GCCCATCCTCTGTGACTCAT | 1 | AGGCCACAGGTATTTTGTCG | 2 |
| MCP-1 | CACTCACCTGCTGCTACTCATT | 3 | TCTGGACCCATTCCTTCTTG | 4 |
| TNF-α | TCTACTGAACTTCGGGGTGA | 5 | CACTTGGTGGTTTGCTACGA | 6 |
| IL-10 | TGCACTACCAAAGCCACAAG | 7 | TGGGAAGTGGGTGCAGTTAT | 8 |
| Arg1 | ATGCTCACACTGACATCAACACTC | 9 | CTCTTCCATCACCTTGCCAATCC | 10 |
| IL-6 | AAGCCAGAGTCCTTCAGAGAGA | 11 | GGAAATTGGGGTAGGAAGGA | 12 |
| RPS29 | TCGTTGGGCGTCTGAAGGCAA | 13 | CGGAAGCACTGGCGGCACAT | 14 |

NfκB Activation

RAW264.7 cells were treated with LPS (200 ng/ml) or SSE (70 μg/ml) for 3 h. NFκB activation was measured using NFkB p65 Transcription Factor Assay Kit (Abcam, Cambridge, UK), according to manufacturer instructions. In this assay, nuclear proteins were extracted using extraction buffer supplied by Abcam. The samples were homogenized and centrifuged at 12,000 RPM for 1 min. The supernatant was collected and protein concentration was measured using the Bradford method. Nuclear protein extract (5 μg) was added to the wells, which were coated with DNA sequence containing the NFkB response element. The selective binding of the transcription factor to its DNA response element was measured according to manufacturer's instructions.

Nitrite Assay

RAW264.7 cells (1×10⁵ cells/ml) were cultured in 6-well plates. After 24 h incubating, NO synthesis was determined by assaying the culture supernatant for nitrite, using Griess reagent (Promega), according to manufacturer instructions.

Western Blot Analysis

RAW264.7 cells were treated with LPS (200 ng/ml) and/or SSE (70 μg/ml) for 24 hr. Protein were lysed using RIPA buffer supplemented with protease and phosphatase inhibitors. The samples were homogenized and centrifuged at 14,000 rpm for 15 min. The supernatant was collected and protein concentration was measured using the Bradford method. Proteins (20 μg per lane) were separated by SDS-polyacrylamide gel electrophoresis and transferred onto nitrocellulose membranes. The membranes were blocked in 5% dry milk solution, incubated with the appropriate antibody solutions (5% BSA in 0.01% TBST) and proteins were immunodetected using the enhanced chemiluminescence method.

Statistical Analysis

Values are presented as mean±SEM. Statistical differences between the treatments and controls were tested by unpaired two-tailed Student's t-test or one-way analysis of variance (ANOVA), followed by Bonferroni's post-hoc testing when appropriate. Analysis was performed using the GraphPad Prism 7.04 software. A difference of p<0.05 or less in the mean values was considered statistically significant.

### Example 1: In Vivo Anti-Inflammatory Effects of S. Spinosum Extract (SSE)

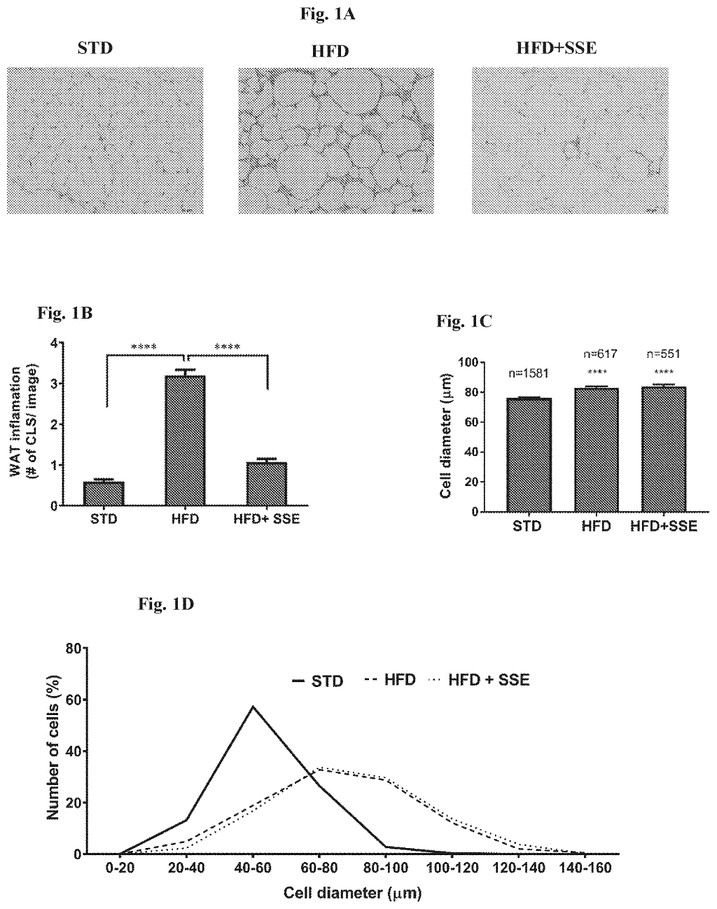

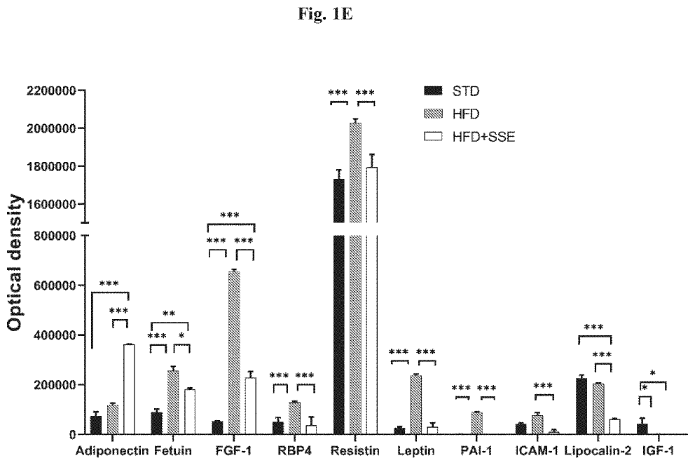

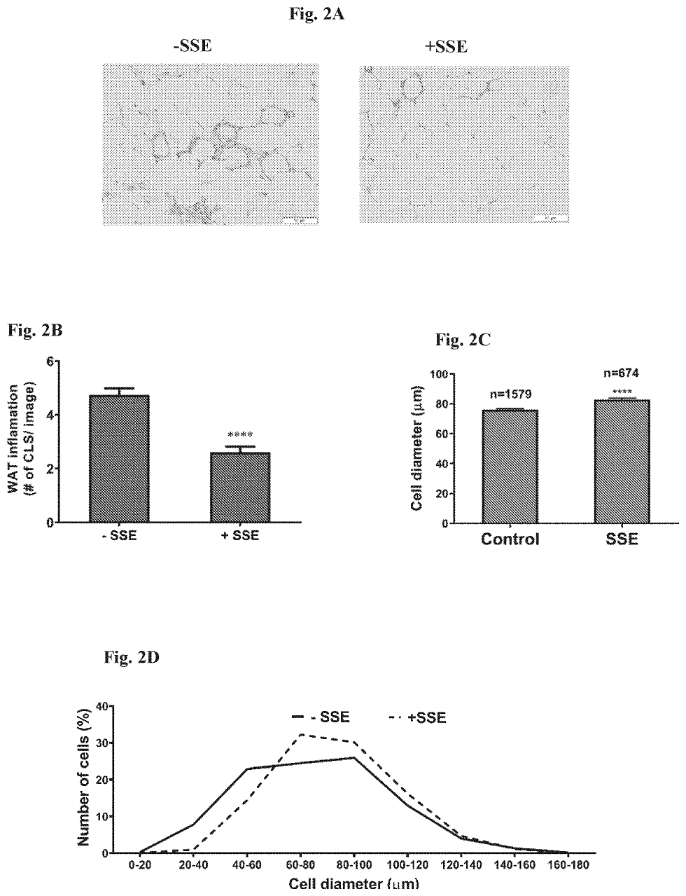

The study was performed on two mouse models exhibiting inflammation: a genetic model of type 2 diabetes (KK-Ay mice) and the diet-induced glucose intolerance model, which develops as a result of HFD feeding. H&E staining of visceral adipose tissue demonstrated the infiltration of leukocytes toward the hypertrophic tissue, as demonstrated by the presence of crown-like structures (CLS) in HFD-fed C57Bl/6J mice and in untreated diabetic KK-Ay mice (FIGS. 1A-1B and 2A-2B, respectively). As can be seen in these figures, SSE reduced the severity of adipose tissue inflammation (as manifested by an increased number of CLS) in both models, although adipocyte diameter was not reduced (number of cells measured was between 500 and 1600), and in KK-Ay mice it was even increased by the treatment (FIG. 1C-1D, 2C-2D, HFD-fed C57Bl/6J mice and untreated diabetic KK-Ay mice, respectively).

Adipocytokine expression of various cytokines was measured in adipose tissue of C57bl/6J mice (FIG. 1E), demonstrating an increase in the expression of Fetuin, FGF-1, RBP-4, Resistin, Leptin and PAI-1 in HFD-fed mice, which was abolished in SSE treated mice. In addition, Adiponectin expression was elevated and ICAM-1 and Lipocalin-2 expression was reduced in SSE treated, compared to HFD-fed mice. The increase in adiponectin expression, which is a known anti-inflammatory agent, along with the reduced expression of ICAM-1, a marker of enhanced leukocyte activation, and of Lipocalin-2, a marker of inflammation, support the anti-inflammatory properties of SSE. The adipocytokine expression profile detected in SSE-treated mice, suggest that SSE induced anti-inflammatory properties.

### Example 2: In Vitro Anti-Inflammatory Effects of S. Spinosum Extract (SSE)

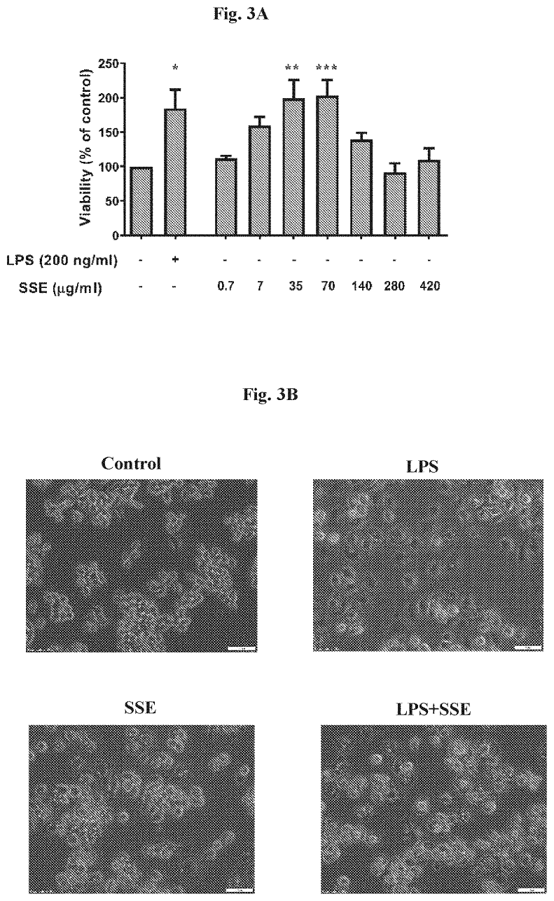

Since SSE was shown above to reduce the inflammatory process in adipose tissues in vivo, in order to verify whether SSE had a direct immunomodulatory function, we investigated its effect on RAW264.7 cells, a macrophages cell line. LPS was used as a positive control to induce a proinflammatory state of the cells. SSE increased proliferation rate in a dose-dependent manner, similar to the stimulatory effect induced by LPS (FIG. 3A). Based on the cell viability assay results, we used SSE at a dose of 70 μg/ml.

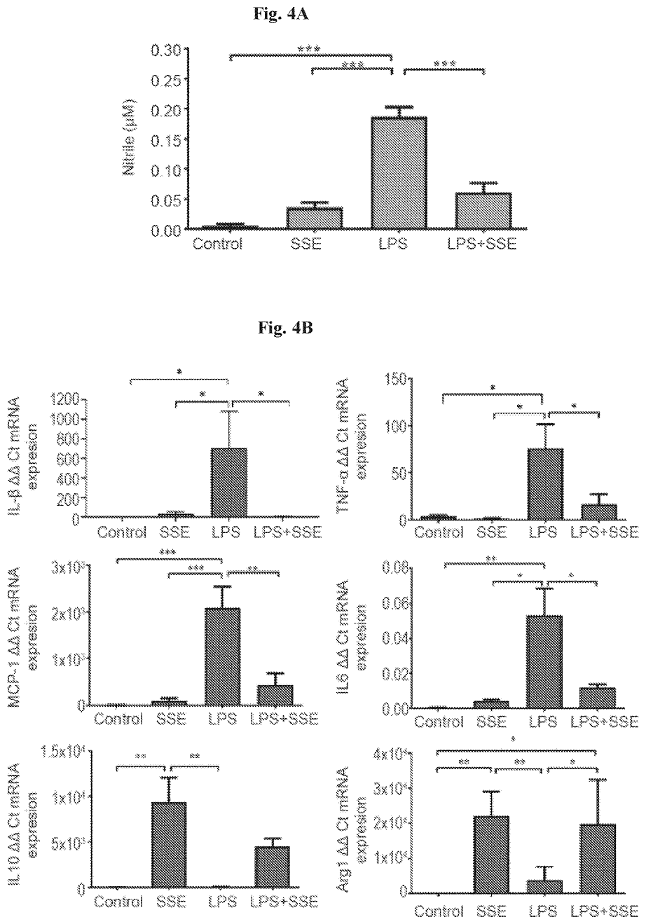

The morphology of cells was affected differently by SSE or by LPS. While the LPS-treated cells were flattened, increased in size and acquired a distinct dendritic morphology, SSE-treated cells underwent fusion and acquired a morphology resembling that of giant multi-nucleated cells (FIG. 3B). However, while both LPS and SSE induced differentiation of the cells, these two agents had a different effect on the specific route of differentiation. LPS increased NO synthesis (FIG. 4A) and mRNA expression of proinflammatory genes (IL1β, TNFα, MCP-1 and IL-6, FIG. 4B), while SSE abrogated this proinflammatory phenotype, and increased the expression of anti-inflammatory genes (IL-10, Arg1, FIG. 4B).

### Example 3: Inflammatory Pathways Activated by SSE

Next, the inventors aimed to clarify the inflammatory pathways activated by SSE. For that, the activation of several proteins, which are known to be involved in distinct inflammatory pathways, was followed: NFκB, MAPK proteins, STAT proteins and Akt. NFκB activation, as measured by the binding of NFκB to its DNA response element, was increased by LPS, while SSE abrogated its activation (FIG. 5A). All 3 members of the MAPK proteins (ERK1/2, JNK and p38) were phosphorylated following LPS treatment (FIG. 5B). SSE also increased the phosphorylation of these kinases. However, its effect on p38 and JNK phosphorylation was lower than the LPS effect. STAT proteins were differently phosphorylated by SSE and LPS. STAT3 was induced to be phosphorylated on its S727 residue by both SSE and LPS, while Y705 was phosphorylated following SSE, but not LPS treatment (FIG. 5C). STAT1 phosphorylation was not affected by these treatments, while STATS phosphorylation was not detected at all (data not shown). Lastly, while Akt phosphorylation was induced by LPS, SSE did not stimulate the phosphorylation of this protein, and also inhibited LPS effect (FIG. 5D).

### Example 4: Immunomodulatory Effect of SSE on Bone Marrow-Derived Macrophages (BMDM)

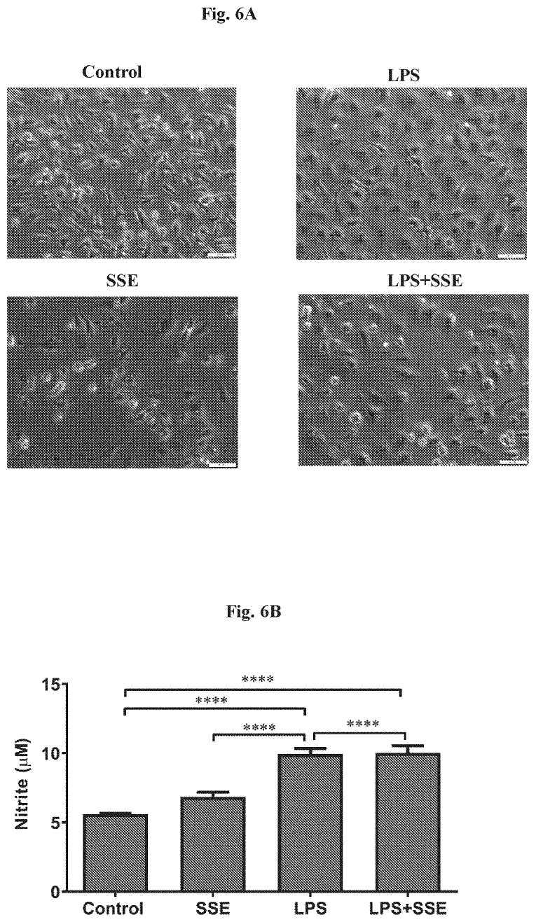

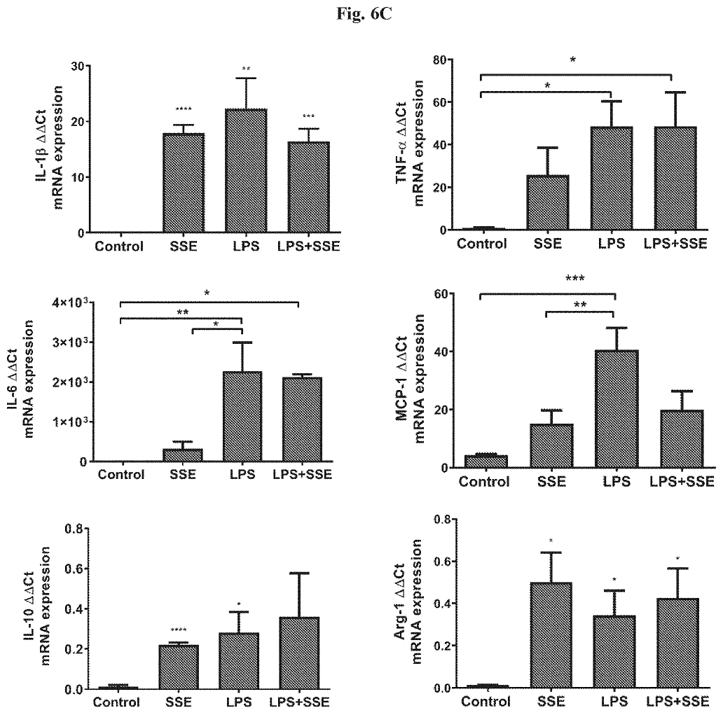

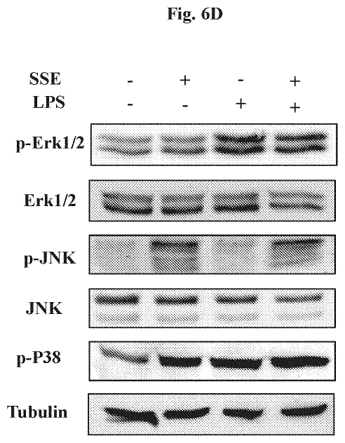

In order to further support the induction of macrophage differentiation by SSE, and its immunomodulatory effect, some of the experiments were validated on a primary culture of BMDM isolated from naïve mice. BMDMs developed a different morphology as a result of treatment by LPS compared to treatment by SSE (FIG. 6A). LPS induced an enlargement of the cells, with a minor vacuolization, while SSE reduced cell density and induced an enlargement and prominent vacuolization of the cells. A combined treatment with both LPS and SSE induced a mixed morphology. NO secretion was significantly increased following LPS treatment, while SSE did not affect basal or LPS-induced NO secretion (FIG. 6B). As shown in FIG. 6C, LPS stimulated mRNA expression of IL1β, TNFα, IL-6 and MCP-1, which are all recognized as pro-inflammatory genes. Interestingly, mRNA expression of IL-10 and Arg1, considered as anti-inflammatory genes, was also increased by LPS treatment. On the other hand, SSE significantly increased mRNA expression of IL1β, but did not significantly affect the expression of the other pro-inflammatory genes measured in this study: TNFα, MCP-1 and IL-6. Expression of IL-10 and Arg1 was significantly stimulated by SSE as well. SSE treatment in the presence of LPS almost did not affect LPS induction of gene expression.

LPS and SSE affect differentially the phosphorylation of MAPK proteins (FIG. 6D). Erk was phosphorylated following LPS stimulation, and JNK was phosphorylated following SSE treatment. P38 phosphorylation was increased by both treatments. A combined treatment of both LPS and SSE induced the phosphorylation of all three MAPK proteins.

### Example 5. Effect of SSE on Inflammation Associated with Alcohol-Related Steatohepatitis in a Mouse Model of Alcoholic Fatty Liver Disease (AFLD)

8 weeks old C57BL/6J female mice are subjected to 6 weeks of chronic ethanol feeding (5%, v/v), with or without the addition of dried SSE at 30, 60 and 90 mg/day. Control mice are fed dextran-maltose instead of the ethanol for replacing the ethanol calories. In the end of the 6 weeks ethanol feeding mice are sacrificed, and livers are perfused, isolated, fixed in 4% paraformaldehyde and embedded in paraffin. Consecutive 4 μm sections are cut and stained with hematoxylin and eosin (H&E). The presence of inflammation and steatosis score is evaluated by a pathologist with Olympus light microscope BX43, Olympus digital camera DP21 with Olympus cellSens Entry 1.13 software.

It is expected that mice fed with ethanol will exhibit inflammation in the liver compared to mice not fed with ethanol. It is further expected that mice treated with SSE will exhibit less liver inflammation compared to untreated mice.

### Example 6: Effect of SSE Administration to Mice on the Activity of Isolated Macrophages

In this set of experiments, we aim to ascertain whether macrophages isolated from mice given SSE show anti-inflammatory activity similar to that seen in macrophages directly treated with SSE, as shown above in Examples 2-4. For this purpose, 8-week-old male BALB/c mice are housed in a temperature- and humidity-controlled pathogen-free animal facility with a 12 h light-dark cycle. SSE is given to mice at doses of 30, 60 and 90 mg/day for 10 days in their drinking water, control mice are not given SSE. Mice are assigned to one of two experiments, as explained below.

The first experiment is directed to examining the effect of SSE treatment on macrophage anti-inflammatory properties following induction of peritonitis and peritoneal macrophage isolation. The thioglycollate-induced peritonitis model is utilized to obtain a high yield of macrophages and to evaluate the effect of SSE on monocyte differentiation. On day 6 after the first SSE treatment, control and SSE-treated mice are injected intraperitoneally with 2 mL of 3.5% sterile thioglycollate. On day 10, thioglycollate-injected mice are sacrificed 1 h after the last SSE treatment. The peritoneal cells are isolated under sterile conditions via peritoneal lavage. After centrifugation, the cells are resuspended and counted. Peritoneal macrophage are cultured in six-well plates overnight at 37° C., and the non-adherent cells are removed. Scavenger receptor activity is determined by measuring the phagocytosis of Alexa Fluor 488-labeled acetylated low-density lipoprotein (acLDL) by cells in flow cytometric analysis. In order to quantify cytokines produced by peritoneal macrophages, the cells are stimulated with 100 ng/mL LPS for 24 h. The supernatant is collected for cytokine measurements by ELISA. Surface markers of M1 and M2 macrophages are detected by flow cytometry.

It is expected that macrophages from mice treated with SSE will show higher anti-inflammatory activity compared to the untreated control.

The second experiment is directed to examining the effect of SSE treatment on the systemic response of intraperitoneal injection of LPS. One hour after the final SSE treatment on day 10, LPS (1.3 mg/kg) is injected intraperitoneally, and after 1 h, the mice are anesthetized, and blood is collected. Serum cytokine level will be analyzed by ELISA.

It is expected that the level of anti-inflammatory cytokines will be higher in serum from mice treated with SSE compared to the control.

### Example 6: Effect of SSE Treatment in a Non-Alcoholic Steatohepatitis (NASH) Model

Methionine/choline deficient diet is used to simulate NASH in mice. However, unlike the manifestation of NASH in humans, this diet causes a reduction in body weight, as well as in liver and adipose tissue weight. Additionally, insulin resistance and glucose tolerance do not develop in spite of the existence of a significant hepatic inflammation. This model therefore corresponds to liver inflammation without metabolic syndrome.

C57Bl/6J Mice at age of 10 weeks are administered either a standard (STD) diet (Envigo, Teklad: TD.2018), or methionine-choline deficient diet (MCD, Envigo, Teklad: TD.90262) with or without the addition of SSE (30, 60, 90 mg/day), for 4 weeks. Mice are euthanized at age 14 weeks, and serum and liver are isolated for biochemical, histological and molecular analyses for the detection of inflammation. It is expected that while liver inflammation is caused by the MCD diet, the SSE will reduce the inflammation.

### Example 7: Effect of SSE Treatment in an Acute Inflammation Model

Carrageenan-induced paw edema is a well-known and widely used model of acute inflammation.

C57Bl/6J mice at age of 10-12 weeks or C57Bl/6J mice pretreated with SSE for 14 days (30, 60, 90 mg/day) are injected with carrageenan (0.1 mL of 1% w/v carrageenan solution) in the hind paw of mouse. In a second set of experiment, SSE (30, 60, 90 mg) is administrated by oral gavage 1 h before, and 1 h after carrageenan injection. The edema that develops after 3-4 hours is estimated by paw weight increase. The inflammation with and without SSE is evaluated by histology and by biochemical analyses of cytokines and mediators of inflammation in the inflamed tissue. It is expected that treatment with SSE will reduce the level of inflammation.

### Example 8: Effect of SSE Treatment in a Colitis Mouse Model

Colitis is induced in C57BL/6 mice at age of 6-8 weeks by the administration of dextran sulfate sodium (DSS) in the drinking water of mice. For chronic DSS-induced colitis, mice are treated with 4 cycles of DSS (1.5%) for 7 days, followed by 10 days of normal drinking water in between each cycle. Control group is given drinking water, without DSS, while SSE-treated groups are given SSE throughout the entire experiment (30, 60, 90 mg/day). Animal are observed for weight, water/food consumption, stool consistency, and the presence of gross blood in feces and at the anus, and Disease activity index (DAI) is calculated by assigning validated scores. At the end of the study mice are euthanized, and their colon is isolated for biochemical, histological and molecular analyses for the detection of inflammation. It is expected that the SSE treatment will reduce colon inflammation.

### REFERENCES

Al-Qura'n, S., Ethnopharmacological survey of wild medicinal plants in Showbak, Jordan. J Ethnopharmacol, 2009. 123(1): p. 45-50.
Ali-Shtayeh, M. S., Z. Yaniv, and J. Mahajna, Ethnobotanical survey in the Palestinian area: a classification of the healing potential of medicinal plants. J Ethnopharmacol, 2000. 73(1-2): p. 221-32.
Bachrach, Z. Y., Ethnobotanical studies of Sarcopoterium spinosum in Israel. Israel Journal of Plant Sciences, 2007. 55(1): p. 111-114.
Elyasiyan, U., et al., Anti-diabetic activity of aerial parts of Sarcopoterium spinosum. BMC Complement Altern Med, 2017. 17(1): p. 356.
Rozenberg, K., et al., Insulin-sensitizing and insulin-mimetic activities of Sarcopoterium spinosum extract. J Ethnopharmacol, 2014.
Rozenberg, K. and T. Rosenzweig, Sarcopoterium spinosum extract improved insulin sensitivity in mice models of glucose intolerance and diabetes. PLoS One, 2018. 13(5): p. e0196736.
Smirin, P., et al., Sarcopoterium spinosum extract as an antidiabetic agent: in vitro and in vivo study. J Ethnopharmacol, 2010. 129(1): p. 10-7.

1. A Sarcopoterium spinosum extract (SSE) for use in preventing or treating inflammation in a subject.

2. The SSE according to claim 1, wherein the inflammation is an acute inflammation.

3. The SSE according to claim 1, wherein the inflammation is a chronic inflammation.

4. The SSE according to claim 1, wherein the inflammation is associated with a disease, disorder or condition selected from bursitis; colitis; cystitis; dermatitis; encephalitis; gingivitis; hepatitis; meningitis; myelitis; nephritis; neuritis; periodontitis; pharyngitis; phlebitis; prostatitis; rhinitis; sinusitis; tendonitis; testiculitis; tonsillitis; urethritis; vasculitis; vaginitis; alcoholic steatohepatitis; an inflammatory bowel disease (IBD) such as ulcerative colitis or Chron's disease; an autoimmune diseases such as asthma, celiac disease, multiple sclerosis, psoriasis, rheumatoid arthritis, or systemic lupus erythematosus; and a metabolic syndrome associated disorder such as insulin resistance, atherosclerosis or nonalcoholic steatohepatitis (NASH).

5. The SSE according to claim 4, wherein the disease, disorder or condition is selected from dermatitis, alcoholic steatohepatitis, ulcerative colitis, Chron's disease, rheumatoid arthritis, and systemic lupus erythematosus.

6. The SSE according to any one of claims 1 to 5, wherein the extract is an extract from the root of S. spinosum.

7. The SSE according to any one of claims 1 to 6, wherein the extract is in liquid form.

8. The SSE according to any one of claims 1 to 6, wherein the extract is in a dry form, such as powder, a tablet or a capsule.

9. A nutraceutical composition comprising a Sarcopoterium spinosum extract (SSE) for use in preventing or treating inflammation in a subject.

10. The nutraceutical composition of claim 9, further comprising other nutritional or dietary supplements and/or one or more pharmaceutically acceptable excipients and/or nutraceutical carriers, diluents, adjuvants, excipients, or vehicles.

11. The nutraceutical composition of claim 9 or 10, formulated for oral administration in the form of a tablet, a capsule, a pill, lozenge or syrup.

12. A method for preventing or treating inflammation in a subject, comprising administering to said subject an extract of Sarcopoterium spinosum (SSE).

13. The method according to claim 12, wherein the inflammation is an acute inflammation or a chronic inflammation.

14. The method according to claim 12, wherein the inflammation is associated with a disease, disorder or condition selected from bursitis; colitis; cystitis; dermatitis; encephalitis; gingivitis; hepatitis; meningitis; myelitis; nephritis; neuritis; periodontitis; pharyngitis; phlebitis; prostatitis; rhinitis; sinusitis; tendonitis; testiculitis; tonsillitis; urethritis; vasculitis; vaginitis; alcoholic steatohepatitis; an inflammatory bowel disease (IBD) such as ulcerative colitis or Chron's disease; an autoimmune disease such as asthma, celiac disease, multiple sclerosis, psoriasis, rheumatoid arthritis, or systemic lupus erythematosus; and a metabolic syndrome associated disorder such as insulin resistance, atherosclerosis or nonalcoholic steatohepatitis (NASH).

15. The method according to claim 14, wherein the disease, disorder or condition is selected from dermatitis; alcoholic steatohepatitis; ulcerative colitis; Chron's disease; rheumatoid arthritis; and systemic lupus erythematosus.

16. The method according to any one of claims 12 to 15, wherein the extract is an extract from the root of S. spinosum.

17. The method according to any one of claims 12 to 16, wherein the extract is in liquid form.

18. The method according to any one of claims 12-16, wherein the extract is in a dry form, such as powder, a tablet or a capsule.
